(19) United States
(12) Patent Application Publication
(10) Pub. No.: US 2015/0231188 A1
(43) Pub. Date: Aug. 20, 2015

(54) MEDICAL FOOD FOR FETAL HEALTH DURING GESTATION

(71) Applicants: John Edgar Menear, Santa Cruz, CA (US); Joseph Ramaekers, Aptos, CA (US)

(72) Inventors: John Edgar Menear, Santa Cruz, CA (US); Joseph Ramaekers, Aptos, CA (US)

(73) Assignee: CORTCONTROL, Santa Cruz, CA (US)

(21) Appl. No.: 14/696,407

(22) Filed: Apr. 25, 2015

Related U.S. Application Data
(63) Continuation-in-part of application No. 14/121,670, filed on Oct. 6, 2014.
Application No. 14/121,670 is a continuation-in-part of application No. 13/999,875, filed on Mar. 29, 2014, now abandoned.
Application No. 13/999,875 is a continuation-in-part of application No. 13/729,923, filed on Dec. 28, 2012.
Application No. 13/729,923 is a continuation-in-part of application No. 12/325,199, filed on Nov. 30, 2008, now Pat. No. 8,357,663.
Application No. 13/999,875 is a continuation-in-part of application No. 13/843,581, filed on Mar. 15, 2013.
Application No. 13/843,581 is a continuation-in-part of application No. 13/718,319, filed on Dec. 18, 2012.
Application No. 13/718,319 is a continuation-in-part of application No. 12/631,745, filed on Dec. 4, 2009, now abandoned.
Application No. 12/631,745 is a continuation of application No. 11/492,464, filed on Jul. 24, 2006, now abandoned.
(60) Provisional application No. 61/964,100, filed on Dec. 24, 2013.

(51) Int. Cl. A61K 35/744; A23L 1/29; A23L 1/30

(52) U.S. Cl. CPC A61K 35/744; A23L 1/3014; A23L 1/296; A23V 2002/00

(57) ABSTRACT

Medical foods that create fertility enhancement also improve the health of a fetus or child. Multiple examples demonstrate this in mammals. To maximize this effect, the mother continues to consume the medical food into the gestation period. Both mother and fetus benefit from higher immunity and lower cortisol. Superior colostrum further builds the child's immune system. Good health as an infant foreshadows health advantages in later life.

### CROSS-REFERENCE TO RELATED APPLICATIONS

This application is a continuation-in-part of U.S. application Ser. No. 14/121,670, filed Oct. 6, 2014, which is currently pending. Application Ser. No. 14/121,670 is a continuation-in-part of U.S. application Ser. No. 13/999,875, filed Mar. 29, 2014, which is currently abandoned. U.S. application Ser. No. 13/999,875 is a continuation-in-part of U.S. application Ser. No. 13/729,923 filed Dec. 28, 2012, which is a continuation-in-part of U.S. application Ser. No. 12/325,199 filed Nov. 30, 2008. U.S. application Ser. No. 13/999,875 filed Mar. 29, 2014 also claims priority to U.S. application Ser. No. 13/843,581 filed Mar. 15, 2013 and currently pending, which is a continuation-in-part of U.S. application Ser. No. 13/718,319 filed Dec. 18, 2012, which is a continuation in part of U.S. application Ser. No. 12/631,745 filed Dec. 4, 2009, which is a continuation of U.S. application Ser. No. 11/492,464 filed Jul. 24, 2006. U.S. application Ser. No. 13/999,875 also claims priority to U.S. Provisional Application 61/964,100 filed Dec. 24, 2013. Application Ser. Nos. 14/121,670, 13/999,875, 13/729,923, 12/325,199, 13/843,581, 13/718,319, 12/631,745, 11/492,464, and 61/964,100 are incorporated entirely by reference into this instant application.

### FIELD OF THE INVENTION

The invention relates to a method of maximizing fetal health during the gestation period, and maximizing child health after birth. In particular, fetal health is advantaged by keeping the mother healthy during the gestation period, and building the immune system of both mother and fetus. The utilized medical food has also demonstrated the ability to control cortisol levels. Upon delivery, the child's health is further advantaged when the mother's colostrum continues to transmit a robust immune system to the child.

Both goals are addressed with a medical food comprising at least transfer factor and lactic acid generating bacteria. The mother consumes the medical food throughout the gestation period, and, where appropriate, after birth.

### BACKGROUND OF THE INVENTION

High human cortisol is defined as cortisol levels that exceed the human population average.

Persistent high cortisol levels correlate with diminished immune capacity. Also, high cortisol compromises general health. One symptom of high cortisol (for male or female) is inability to conceive a child. A second symptom is sub-optimal child health at delivery. In this context, metrics for sub-optimal health include (1) a weak immune system, (2) susceptibility to infection, (3) frequent flus or colds, (4) low energy level, (5) high stress due to discomfort, and (6) slower-than-normal growth. This metric list is not complete.

In the United States, the number of women ages 15-44 with impaired fecundity (impaired ability to get pregnant or carry a baby to term) is 6.7 million. Multiple fertility clinics exist to help women with fertility issues.

Research indicates that stress is an important factor. Speculation about the correlation between stress and infertility is part of the prior art.

One fertility clinic broadcasts the following message: “The best fertility treatment is beating stress”. A 2009 study at Emory University concluded that “We think there are women who have sub-clinical forms of stress and who are infertile as a result . . . . ” More work on cortisol correlation was suggested in this Emory study. The study is informative, but does not recite a treatment method with transfer factor and lactic acid generating bacteria.

Although cortisol reduction and immune enhancement have long been proposed as a way to enhance human fertility, no human-specific treatment method utilizing at least transfer factor and lactic acid generating bacteria had been fully enabled prior to application Ser. No. 13/999,875 (Menear and Ramaekers, filed Mar. 29, 2014.

Prior animal studies performed by Ramaekers demonstrate that the benefit of the fertility medical food goes beyond fertility enhancement. Early stage miscarriages were reduced. Also, livestock off-spring was healthier and experienced a higher survival rate when the mother consumed the medical food during gestation.

A healthy child is a universally desirable goal. Miscarriages, infant mortality, and sick children are heart-breaking outcomes.

Although methods of administration differ between animals and humans, the same benefits of a medical food are available to humans. Fertility is enhanced, the fetus develops inside a healthy mother, and the child begins life in a healthy state.

There is a need to provide a medical food and a method of use that enhances the health of fetus and child.

### BRIEF SUMMARY OF THE INVENTION

The instant invention is a method of maximizing fetal and child health with a medical food that is consumed by the mother. Medical food consumption may be started at several points on a timeline.

For example, the mother may choose to start medical food consumption 3-100 days before conception as part of an enhanced fertility program. Starting at 100 days prior to conception offers the advantage that the egg is fully matured in the presence of the medical food.

If consumption is not practical before conception, the mother's consumption may begin during gestation. In this case, the quality of fertilized egg is not advantaged, but the developmental environment is still advantaged.

A similar recommendation exists when in-vitro-fertilization is needed.

In cases of tubular blockage, consumption before in-vitro-fertilization (IVF) is recommended as part of the fertility enhancement program. In this case, medical food consumption 3-100 days before IVF is desirable for both male and female. This means that the child grows from a healthy egg and healthy sperm—plus gestation occurs in an optimal environment.

If consumption before in-vitro-fertilization is not practical, consumption still confers benefit. Again, consumption during gestation allows the fertilized egg to develop in an advantaged environment.

The basic medical food to promote fetal and child health is a combination of transfer factor and lactic acid generating bacteria. Adding glucans to the combination of transfer factor and lactic acid generating bacteria has proven to be particularly effective for mammals due to a synergistic effect. Humans are mammals, and the same benefits are expected.

This instant invention is a method of supporting fetal and child health with a food that (1) reduces human cortisol levels, (2) builds the immune system of child and mother, and (3) balances endocrine function.

The medical food contains at least transfer factor and lactic acid generating bacteria at defined dosage levels. A medical food composition that includes consumption of transfer factor and lactic acid generating bacteria is within the scope of this invention. A medical food composition that does not include consumption of transfer factor and lactic acid generating bacteria is outside the scope of this invention. This medical food may include other components in conjunction with transfer factor and lactic acid generating bacteria.

One probable biochemical mechanism involves the mother's increased progesterone levels. Progesterone means “for gestation”. Progesterone is necessary for healthy egg production, and the egg's stable attachment to the uterus. Progesterone is further necessary to feed and nourish the uterus during pregnancy, and, hence, support the fetus. Low progesterone is associated with early miscarriage and failure to reach full term.

However, this application is not limited by any specific biochemical mechanism. Claims are based on actual experience with mammals. Claims are based on animal data rather than theory.

Objects of this invention include some or all of the following: (1) a method to increase fetal or child health by starting with a healthy egg and sperm, (2) a method to increase fetal or child health via gestation within a healthy mother, (3) a method to increase the quality of a mother's colostrum, (4) a method to increase child health by providing a high quality colostrum at birth, (5) a method to increase child health by building a robust immune system, and (6) a method to increase child health by minimizing the incidence of childhood diseases.

There are two medical food compositions used. The first is a combination of transfer factor and lactic acid generating bacteria. This composition is encompassed by Ramaekers' domineering U.S. Pat. No. 6,962,718, claim 6. The second is a combination of transfer factor, lactic acid generating bacteria, and glucans. Glucans may be present as mushrooms. This composition is also encompassed by Ramaekers' domineering U.S. Pat. No. 6,962,718, and contains one additional limitation (glucans).

Food compositions may be augmented with additives other than transfer factor, lactic acid generating bacteria, and glucans. The use of additives remains within the invention scope, providing that (at least) transfer factor and lactic acid generating bacteria are present.

Dosages of medical foods are adjusted for patient weight. This is described by Ramaekers in Application Publication 20090170774, which arises from a priority application. At the bottom of Table 1 in Application Publication 20090170774, amounts are calculated for livestock weighing over 450 pounds and cats/dogs weighing as little as 8 pounds.

Consumption frequency for animals is usually fixed at once per day or once every second day.

Humans will respond to dosage levels and consumption frequencies that mimic animal use. But, there are better options. Recommended human treatment durations and human frequency of consumption differ from other mammals. And optimal human treatments allow adjustment based on continual cortisol measurement or T-cell measurement. Alternately, a health care professional may recommend a dosage change, based on other metrics.

Human administration frequency has a wider range than other mammals. Human consumption varies between five times per day and once per week. For livestock, consumption is typically fixed at once per day or once every second day.

The relative proportion of transfer factor, lactic acid generating bacteria, and glucans within a human dose may vary. Although typical proportions can be borrowed from animal testing (per Ramaekers in U.S. application Ser. Nos. 13/729,923 and 13/843,581), proportions may be modified to best serve each individual. Humans can imagine stressful situations, which stimulates cortisol production—just like a real stressful situation. Hence, the variation of response time is wider. Analytical feedback from cortisol measurements may be used to trigger proportion modification.

Some clients consume transfer factor, lactic acid generating bacteria, and glucans together. For other clients, transfer factor, lactic acid generating bacteria, and glucans are taken at different times during the day or week. Component separation and consumption at different times are within the scope of this invention. Separate consumption and was recited in Ramaekers' U.S. Publication 20070128253, which benefits this application. The human body performs the mixing.

### BRIEF DESCRIPTION OF THE DRAWINGS

FIG. 1 shows a positive correlation between conception rate and off-spring survival rate due to the invented medical food. The medical food is a positive factor for both. Note that the medical food was consumed before conception and during gestation. Healthier eggs and sperm lead to a healthier embryo. A healthier embryo relates to the quality of health after birth.

FIG. 2 shows a dramatic increase in embryo quality due to consumption of the medical food. The difference between TEST and CONTROL cows was statistically significant at the 99.9% level.

FIG. 3 graphically shows that the medical food simultaneously led to a better survival rate and a lower weaning time. Faster weaning is considered an indicator of good health. The same advantages are available for humans, providing that medical food consumption continues into gestation.

FIG. 4 shows that reduced cortisol levels are one of the mechanisms operating to the child's advantage. However, this invention is not limited to any specific mechanism.

### DETAILED DESCRIPTION OF THE INVENTION

This invention is a method-of-use for a specific medical food. It uses a medical food to optimize fetal and infant health. The medical food is consumed by the mother during gestation. It may be continued after delivery to produce highly effective colostrum, which leads to a superior immune system for the infant.

During the gestation period, the mother's immune system is supported. Hence, the mother is inherently healthier. Fewer colds, flus, and general diseases are predictable. Further, the medical food controls cortisol (the main stress hormone) levels in the shared blood supply. The combination of robust immunity and low stress provide the best conditions for healthy fetal development.

Robust immunity and controlled cortisol are cited as biochemical mechanisms, and other positive mechanisms may contribute. However, this invention is not limited in scope by any proposed mechanism. Support for this invention is found in documented data for animals, rather than in theories or hypotheses.

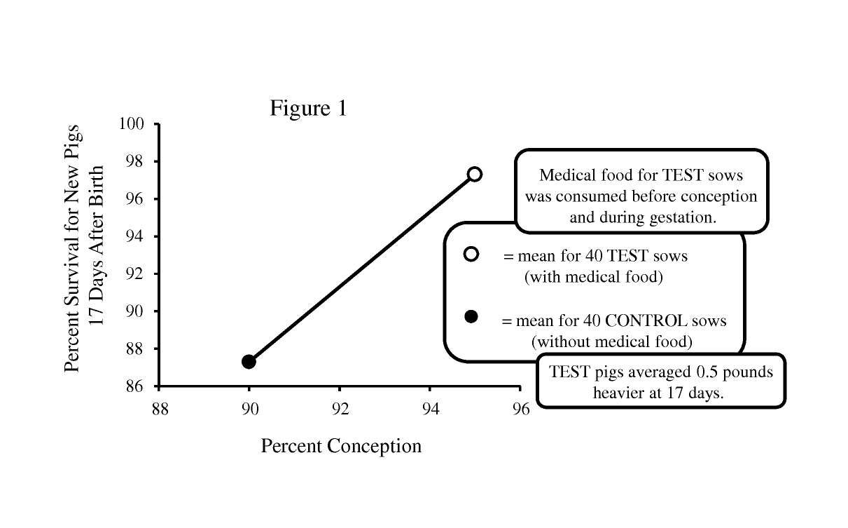

FIG. 1 is a correlation-type graph that addresses the essence of the current invented method. It shows that a medical food for fertility enhancement should be continued into the gestation period to optimize off-spring health. The medical food increased the conception rate from 90 to 95%, and increased the survival rate of young pigs from 87.3 to 97.3 percent. The core theme is that conception rate and survival rate increased simultaneously. The results shown in FIG. 1 are typical, and represent a wide scope of testing. The importance to human fertility is very important because of the today's tendency to delay child bearing. A method that supports maternal health also supports fetal health.

The medical food used for this method includes (1) at least transfer factor and lactic acid generating bacteria, or (2) at least transfer factor, lactic acid generating bacteria, and glucans. Dosage consumption frequency is normally between five times per day and once per week. The ingredients in each dosage may be separated for consumption. If they are separated, each essential ingredient should be consumed between five times per day and once per week.

Transfer factor is present in each dosage between 0.05 to 50 mg per pound of mother's body weight.

Lactic acid generating bacteria in each dosage is present at 0.47 to 10 mg per pound of mother's body weight, providing that the lactic acid generating bacteria has a live count of 1.5 to 3.5 million colony forming units per ounce.

Glucans in each dosage are present at 0.1 to 10 mg per pound of mother's body weight.

Transfer factor is produced by leucocytes and lymphocytes. Transfer factor comprises small water soluble polypeptides of about 44 amino acids that stimulate or transfer cell mediated immunity from one individual to another.

Alternative sources of transfer factor include avian transfer factor, ova transfer factor, and colostrum from goats, pigs, horses and humans. This listing is not complete. In addition, combinations of transfer factors from any number of sources may be used in formulations.

The properties, characteristics and processes for obtaining transfer factor or transfer factors are discussed in U.S. Pat. Nos. 4,816,563; 5,080,895; 5,840,700, 5,883,224 and 6,468,534, the contents of which are hereby incorporated by reference into the present application.

In certain embodiments, substantially purified transfer factor has a molecular weight of less than 10,000 Daltons.

Transfer factor is commercially available, and known to be safe for short or long term use.

Lactic acid generating bacteria is an important component of the pertinent medical foods, and is GRAS (generally recognized as safe). Lactic acid generating bacteria support digestion and brain health. Lactic acid generating bacteria provide healthful effects that are found in non-pasteurized sauer kraut and cod liver oil. Within the intestinal tract, lactic acid generating bacteria are beneficial. It has been estimated that 80% of human health depends on beneficial intestinal bacteria.

A human body becomes stressed by poor digestion, and cortisol levels will reflect that stress. Lactic acid generating bacteria helps reduce cortisol via improved digestion.

Glucans (polysaccharides) are known to support the immune system. When combined with transfer factor and lactic acid generating bacteria, a synergy is created. The combined effect on health is greater than the effect predicted from summing the individual components.

There are connections between fertility enhancement and off-spring health. For example, when a cow consumes the medical food to get pregnant, (1) the calf has a better survival probability, (2) the calf gains weight faster than normal, (3) the calf's coat is shinier and fuller, and (4) veterinarians assess overall symptoms of health as superior. The comparison is to control groups that do not receive the medical food.

Positive fertility-and-survival correlations hold true for pigs, sheep, horses, and other mammals. Results on humans should parallel other mammals. The same food-based strategies that lead to higher conception rates also support the child's health and survival probability.

History suggests that early in life, the benefits of the medical food are particularly valuable. Avoiding childhood diseases sets the stage for good health throughout life. In contrast, repeated childhood diseases foreshadow problems in later life.

Examples of optimal infant health are drawn from animal studies. Following are some examples in abbreviated form. More detail is contained in the priority documents that are incorporated by reference. Those details have no cumulative value, and will not be repeated here.

Two hundred heifers were divided into 4 groups of 50. Groups 1-3 were fed (or injected with) a medical food composed of transfer factor, lactic acid bacteria and glucans. Group 4 was a control. The heifers from cows receiving the medical food gained more weight and showed lower morbidity, compared to heifers from cows that didn't receive the medical food. Results were significant at the 95% confidence level.

One hundred calves in Fort Bidwell, Calif. with dysentery were divided into 2 groups. Forty (40) TEST calves were treated with a medical food composed of transfer factor, lactic acid bacteria and glucans. Sixty (60) calves were CONTROLS, and did not receive the medical food. The treated calves recovered from dysentery, gained weight, and are healthy members of the herd. The control calves remained sick, but were finally given medical food and recovered. This example shows the importance of building immunity at an early age. One way to build human immunity is to provide a medical food to the mother during gestation. The medical food supports the mother's health, and provides superior colostrum to the child at birth.

Twenty young kittens were suffering from feline pneumonitis, and were not responding to a daily dosage of Clavamox. So, treatment with clavamox was halted upon initiation of the medical food feeding. After five days on the medical food, all 20 kittens appeared symptom free of the pneumonitis. Again, the effect of a medical food on young mammals is clear. If the mother, had consumed the medical food during gestation, there is a good chance that the kittens would not have developed the pneumonitis. And, if the kittens from a treated mother were to develop pneumonitis, the mother's colostrum would promote a quick recovery.

In a parallel study, an animal shelter had 80 sick kittens suffering with feline pneumonitis. The prior incidence of morbidity was 80% with 10% death. The administration of a medical food for 7 days reduced the morbidity to 30% and mortality to 5%. The ideal case would have been administration of the medical food to the mother cats before birth.

A farm in Pennsylvania had 40 ovum donor cows that were losing most of their calves. Many of the adult cows were ill. The University of Ohio diagnosed the cows and calves as suffering from Clostridium Perfrengens type A. The morbidity rate for the calves was 100% and mortality was 80%. After initiation of a medical food program, no dysentery was observed in the herd, and no more calves have died. This example demonstrates how an unhealthy mother correlates to sick off-spring. Keeping the mother healthy is important to child health, and consuming a medical food during gestation supports good child health.

Fifty (50) mares and foals at a farm in Yakama Wash. were infected with Rhodococcus. Treatment with Azithomycin proved ineffective. A study was then conducted in which foals weighing approximately 110 pounds each were divided into TEST and CONTROL groups. The TEST group received medical food; the CONTROL group did not. The TEST foals developed an average white blood count of 13,000. The CONTROLS developed an average white blood count of 8,000. Clearly, the TEST foals had a much higher level of immune readiness. Rhodococcus symptoms for the TEST foals were reduced by 60% within 5 days, compared to the CONTROL foals. Afterward, all foals were weighed weekly, and the TEST foals were consistently heavier. If the mares had consumed medical food during gestation, it is probable that neither mares nor foals would have had Rhodococcus at the time of birth.

An outbreak of PURRS (porcine upper respiratory and reproductive disease in swine) occurred at a swine operation located in Iowa. The outbreak affected thousands of young pigs with high mortality and morbidity rates. A set of 6200 pigs, weighing about 12 pounds each, that had just arrived from the farrowing house, and were in danger of developing PURRS. After 10 days of medical food consumption, all the young pigs appeared normal with no indication of PURRS. Young mammals respond well when the immune system is optimized. In a parallel manner, a human child's immune system is inherently optimized if born to a mother consuming medical food during gestation.

Another field study involved young 132 pigs entering the nursery at a swine farm in Ohio. The goal of the study was to evaluate the effect of a medical food on general health for the new pigs. Those pigs were divided into 66 TEST and 66 CONTROL pigs. All the pigs were fed the same diet. TEST pigs received the medical food; CONTROL pigs did not. After 14 days the TEST pigs appeared disease free and exhibited better weight gain than the CONTROL pigs. Upon slaughter the carcass data shows no liver and kidney disease in the TEST group but around a 1.5% disease rate in the CONTROL group.

A second pig operation had a serious outbreak of PURRS disease affecting 60% of the young pigs. These young pigs also had many other diseases due to the complexity of PURRS. Five thousand new young pigs were scheduled to arrive in one week, and the farm wanted to reduce the infection rate. So prior to delivery each new pig was given a medical food before leaving the mother. Medical food consumption was also continued for 3 more days after reaching the nursery. At the end of this protocol, morbidity for the new pigs was reduced 20%. Their better developed immune system (from both medical food consumption and their mother's colostrum) protected them from developing PURRS.

In the cattle industry, fertilized ova are flushed from the uterus of a donor cow and subsequently implanted in a recipient cow. A similar technique is used for humans: in-vitro-fertilization. The difference is that, with human in-vitro-fertilization, the embryo is implanted back into the same female donor. When blocked tubes are involved, in-vitro-fertilization is a standard technique.

All embryos are not equal, and Oklahoma State University has a grading system for embryos. The 4 grades are (a) grade 1, excellent or good; (b) grade 2, fair; (c) grade 3, poor; and (d) grade 4, dead or degenerating. Grade 1 embryos are more likely to develop from an egg and sperm matured in the presence of a fertility enhancement medical food. And a child that develops from a grade 1 embryo is more likely to experience good health than a child from a grade 2 or 3 embryo. The healthy infant is positioned for the maximum quality of life during juvenile and adult years.

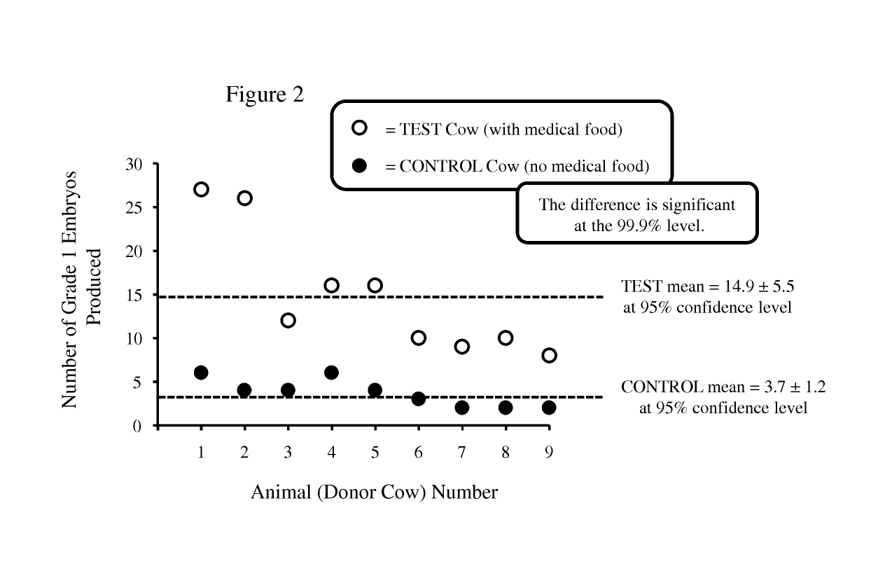

FIG. 2 shows the increase in grade 1 cattle embryos produced by a medical food. Grade 1 embryos are more robust than grades 2-4, and more likely to survive implantation. The human case is analogous when in-vitro-fertilization is used. Maturing of quality eggs and sperm before conception is an important contributor to off-spring health.

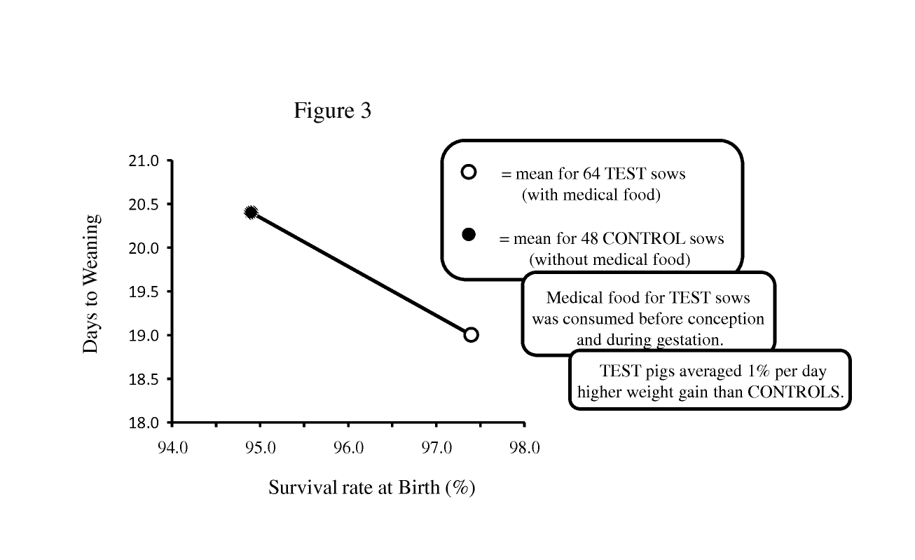

FIG. 3 shows that sows (challenged with a serious flu virus during pregnancy) treated with medical food produced healthier young pigs. The comparison is to untreated sows. The percent of pigs born dead-on-arrival was cut in half by the medical food. The average number of days until weaning was reduced from 20.35 to 18.97 by the medical food. And, the average weight gain increased due to the medical food. This data further demonstrates the value of medical food consumption by the mother during gestation.

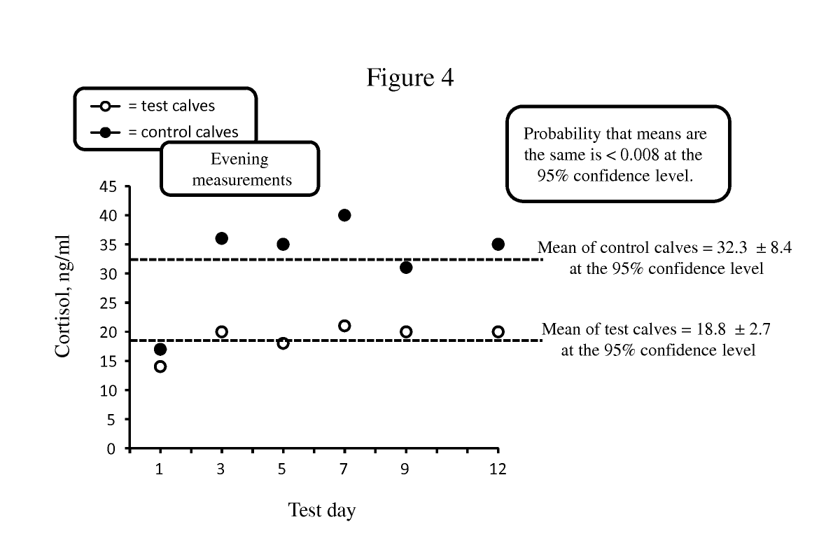

FIG. 4 shows the effect of the medical food on cortisol levels. Cortisol is the primary stress hormone, and plays an important role in fertility and general health. Cortisol is correlated to multiple diseases.

The invention claimed is:

1. A food-based method for supporting the health of fetus during gestation comprising:
combining transfer factor transfer and lactic acid generating bacteria to create a medical food, wherein
said transfer factor includes some polypeptides with a molecular weight below 10,000 Daltons;
feeding a mother said medical food during a fraction of said gestation period or during the entire said gestation period;
choosing a dosage level for said medical food based on said mother's weight, and
selecting a frequency of said feeding between five times per day and once per week,
wherein each said feeding comprises one said dosage level.

2. The method of claim 1 wherein glucans are added to said medical food.

3. The method of claim 2 wherein said glucans are derived from natural or hybrid mushrooms.

4. The method of claim 2 further comprising measuring cortisol levels periodically.

5. The method of claim 4 further comprising adjusting said dosage levels or said frequency based on said cortisol levels.

6. The method of claim 2 further comprising measuring killer T-cells levels periodically.

7. The method of claim 6 further comprising adjusting said dosage levels or said frequency based on said killer T-cells levels.

8. The method of claim 2 further comprising measuring said fetus' vital signs periodically.

9. The method of claim 8 further comprising adjusting said dosage levels or said frequency based on said vital signs.

10. The method of claim 2 further comprising changing the relative proportions of transfer factor, lactic acid generating bacteria, and glucans included within said medical food.

11. The method of claim 2 wherein said transfer factor in each said dosage is present at 0.05 to 50 mg per pound of said mother's body weight.

12. The method of claim 2 wherein said lactic acid generating bacteria in each said dosage is present at 0.47 to 10 mg per pound of mother's body weight.

13. The method of claim 12 wherein said lactic acid generating bacteria has a live count of 1.5 to 3.5 million colony forming units per ounce.

14. The method of claim 2 wherein said glucans in each said dosage is present at 0.1 to 10 mg per pound of mother's body weight.

16. A method for supporting the health of a mother's new-borne infant comprising:
combining transfer factor transfer and lactic acid generating bacteria to create a medical food, wherein
said transfer factor includes some polypeptides with a molecular weight below 10,000 Daltons;
feeding said mother said medical food before or during the period of colostrum production;
choosing a dosage level for said medical food based on said mother's weight; and
selecting a frequency of said feeding between five times per day and once per week,
wherein each said feeding comprises one said dosage level.

17. The method of claim 16 wherein glucans are added to said medical food.

18. The method of claim 17 wherein said transfer factor in each said dosage is present at 0.05 to 50 mg per pound of said mother's body weight.

19. The method of claim 17 wherein said lactic acid generating bacteria in each said dosage is present at 0.47 to 10 mg per pound of mother's body weight.

20. The method of claim 19 wherein said lactic acid generating bacteria has a live count of 1.5 to 3.5 million colony forming units per ounce.

21. The method of claim 17 wherein said glucans in each said dosage is present at 0.1 to 10 mg per pound of mother's body weight.

22. A method for supporting the health of an infant comprising:
combining transfer factor, lactic acid generating bacteria, and finely divided vegetables, meats, or fruits to create a baby food, wherein
said transfer factor includes some polypeptides with a molecular weight below 10,000 Daltons;
feeding said infant said baby food after birth when solid food is normally consumed;
choosing a dosage level for said transfer factor and said lactic acid generating bacteria within said baby food based on said infant's weight; and
selecting a frequency of said feeding of said baby food between one and eight times per day,
wherein each said feeding comprises one said dosage level.

23. The method of claim 22 wherein glucans are added to said baby food.
